(19) United States
(12) Patent Application Publication
(10) Pub. No.: US 2019/0127775 A1
(43) Pub. Date: May 2, 2019

(54) METHODS FOR WHOLE CELL ANTIBACTERIAL ACTIVITY SCREENING AND BIOCHEMICAL TARGET IDENTIFICATION

(71) Applicant: CURATORS OF THE UNIVERSITY OF MISSOURI, COLUMBIA, MO (US)

(72) Inventor: William G. Gutheil, KANSAS CITY, MO (US)

(73) Assignee: CURATORS OF THE UNIVERSITY OF MISSOURI, COLUMBIA, MO (US)

(21) Appl. No.: 16/094,093

(22) PCT Filed: Jan. 10, 2017
(86) PCT No.: PCT/US17/12820

§ 371 Date: Oct. 16, 2018

Related U.S. Application Data
(60) Provisional application No. 62/326,440, filed on Apr. 22, 2016.

(51) Int. Cl. C12Q 1/18

(52) U.S. Cl. CPC C12Q 1/18

(57) ABSTRACT

Disclosed are devices and methods for preparing pre-metabolized compound libraries and for screening compound metabolites in a high throughput format. The devices and methods disclosed herein increase the chemical diversity of a chemical library prior to screening, and thereby enhance the number and value of hits identified in such chemical screening efforts.

### CROSS-REFERENCE TO RELATED APPLICATIONS

This application claims priority to U.S. Ser. No. 62/326,440, filed on Apr. 22, 2016, the disclosure of which is incorporated by reference in its entirety.

### BACKGROUND OF THE DISCLOSURE

The present disclosure relates generally to drug and bioactive agent discovery. More particularly, the present disclosure relates to devices and methods of drug discovery by pre-metabolizing a chemical compound and analyzing the compound metabolite(s) for active compound metabolites using a suitable assay. Pre-metabolization can be performed using any of a wide variety of cells, cell lysates, cell fractions, and enzymes; from a wide variety of organisms including human, monkey, mouse, rat, dog, bird, any multicellular or single cell eukaryote, bacteria, or archaea; individually or in combination. The devices and methods increase the chemical diversity of a library prior to screening, and thereby enhances the number and value of hits identified in such screening efforts.

The screening of chemical compound libraries has become an essential component of the drug discovery process. In this approach, collections (libraries) of chemical compounds are screened for activity using some sort of assay for the desired activity. Compounds showing activity (hits) can then be further investigated and refined to provide new drug leads, and ultimately new drugs. This approach is now widely applied for pharmaceutical industry drug discovery efforts, and in academic drug and bioactive agent discovery efforts. High throughput screening (HTS) typically refers to large-scale screening efforts of up to millions of compounds, which are performed in highly automated HTS screening facilities. Most medium to large pharmaceutical companies have HTS screening facilities, as do a number of academic institutions. The screening of large numbers of compounds typically uses microtiter plates to store compounds in a format that allows large numbers of compounds to be conveniently handled by automated machines, and uses microtiter plate based assay methods and detectors for eliciting and measuring the desired assay activity on large numbers of samples.

Essential to the library screening approach for the identification of drug leads and bioactive agents is the availability of suitable chemical compound libraries for screening. A key feature of chemical compound libraries is their chemical diversity, which refers to the chemical space encompassed by the library. Substantial effort has been devoted to developing chemical libraries with increased and unique chemical diversity. Most medium to large pharmaceutical companies have their own large sets of proprietary compound collections, and academic screening facilities use commercial libraries as well as compounds generated through programs such as the NIH supported Chemical Methodologies and Library Development (CMLD) program. There are many commercial and public libraries available, for most of which the chemical compound identities are publically known. For example, Selleckchem (Houston, TX) offers a number of screening libraries, most of which are targeted towards a specific type of biological activity, ranging in size from <50 compounds (anti-diabetic compound library) to >5000 compounds, including an FDA approved drug library of 1400 compounds and a natural products library of 133 compounds. Maybridge (Leicestershire, United Kingdom) offers diversity oriented screening libraries of 14,000-53,000 compounds. ChemBridge (San Diego, CA) offers diversity libraries ranging from 10,000-620,000 compounds. TargetMol (Boston, MA) offers a variety of specialized libraries ranging in size from a 41 compound human endogenous ligand library to a 1700 compound approved drug library.

The aforementioned libraries are comprised of pure or nearly pure compounds (where each compound is generally >95% pure). Libraries can also be comprised of mixtures of compounds. For example, several sources offer libraries comprised of crude extracts from natural sources, including Cyano Biotech (Berlin, Germany), GreenPharma (Orleans, France), and through the National Cancer Institute's Developmental Therapeutics Program (Bethesda, Md.).

Nearly all drugs and other xenobiotics are transformed into at least one metabolite, and generally more. For example, indinavir has 13 identifiable metabolites and breviscapine has 10. Such metabolites frequently have distinct biological activities. In Domagk's classic studies leading to the identification of prontosil as an antibacterial agent, it was fortuitous that antibacterial activity screening was performed in live mice, since protosil is inactive in vitro, and is only active after metabolic conversion into the active agent: sulfanilamide This fortuitous discovery led to the entire class of sulfanilamide antibacterial agents.

Approaches to identifying new active agents (hits) using compound libraries include enzyme or receptor specific screening and whole cell screening. Assays are typically performed in microtiter plates (24, 96, 384, 1536, or 3456 well plates). Chemical compounds from a chemical compound library are added to the wells in these plates. To each well of the plate is also added some biological entity to be screened for activity against. These biological entities are typically a purified protein (enzyme or receptor), or a type of cell (bacterial cells, cancer cells, etc.), or an animal tissue or embryo. After some incubation time has passed to allow the biological matter to absorb, bind to, or otherwise react (or fail to react) with the compounds in the wells, measurements are taken across all the plate's wells, either manually or by a machine. Manual measurements may be necessary if microscopy is used to (for example) seek changes or defects in embryonic development caused by the wells' compounds, or when looking for effects that an automated system could not easily determine. Otherwise, automated analysis instruments can be used assess the wells using spectroscopic methods such as UV-vis absorbance or fluorescence measurements. In this case, the output of the library screen is a grid of numbers, with each number mapping to the value obtained from a single well. A high-capacity analysis machine (e.g. a microplate reader) can measure tens of plates in the space of a few minutes, generating thousands of experimental data points very quickly. This data can then be processed to identify which wells containing active compounds (hits).

Chemical compound library screening is currently an important aspect of many drug and bioactive agent discovery efforts. Library chemical diversity is a key determinant of the value and success of such efforts, and novel approaches to increasing library diversity will benefit such efforts. Accordingly, there is a need to increase chemical diversity.

### BRIEF DESCRIPTION OF THE DISCLOSURE

The present disclosure is generally directed to chemical compound library screening for drugs, drug leads, and bioactive agents. More particularly, the present disclosure relates to the use of pre-metabolized compound libraries to enhance the success rate of the screening process, methods for preparing pre-metabolized compound libraries, methods for screening a pre-metabolized compound library to identify library compounds with active metabolites, and methods to identify active metabolites.

In one aspect, the present disclosure is directed to a pre-metabolized compound library comprising: a plurality of compound metabolites; and a substrate, wherein the plurality of compound metabolites are selectively distributed in a plurality of wells of the substrate.

In another aspect, the present disclosure is directed to a method for preparing a pre-metabolized compound library. The method comprises providing a compound library comprising a plurality of compounds; and contacting the plurality of compounds with a metabolizing agent to generate a plurality of compound metabolites.

In another aspect, the present disclosure is directed to a method for screening a pre-metabolized compound library to identify active metabolites of a compound library. The method comprises providing a compound library comprising a plurality of compounds; contacting the plurality of compounds with a metabolizing agent to generate a plurality of compound metabolites; contacting the plurality of compound metabolites with a target; and analyzing the target.

In another aspect, the present disclosure is directed to a method for screening a pre-metabolized compound library to identify compounds active as antibacterial agents as their metabolites. The method comprises providing a compound library comprising a plurality of compounds; contacting the plurality of compounds with a metabolizing agent to generate a plurality of compound metabolites; contacting the plurality of metabolized compounds at a suitable concentration with a dilute bacterial culture in the presence of suitable media for bacterial cell growth; incubating the mixture under conditions favorable to bacterial cell growth; and analysis of the resulting mixture for bacterial cell growth or growth inhibition by an appropriate means, such as by measuring culture turbidity by UV-vis absorbance at 590 nm. Comparison with a parallel screen performed with the un-metabolized library allows compounds active as their metabolites to be identified.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustration depicting the Pre-met Library Screening Concept. Compounds in an un-metabolized (“Un-Met”) library are metabolized to generate the Pre-Metabolized (“Pre-Met”) compound library. Compounds of the Un-met and Pre-Met libraries are then screened for activity against a suitable target, giving Un-Met library hits (actives), and Pre-Met library hits (actives). Compounds showing activity in the Pre-Met library screen but not in the Un-Met library screen are compounds active as their metabolites.

FIG. 2 is a Venn diagram outlining the realm of possibilities anticipated for a Pre-Met library screen performed in parallel with a corresponding Un-Met library screen. Region descriptions: 00—UM and PM wells inactive, Parent compound(s) and compound metabolites inactive; 01—UM well active, PM well inactive, Parent compound(s) active, but activity lost during metabolism resulting in inactive compound metabolite(s); 10—UM well inactive, PM well active, Parent compound(s) inactive, but has at least one active compound metabolite; 11—UM and PM active, Parent compound(s) active and not metabolized, or Parent compound(s) active and metabolized to active compound metabolite(s).

FIGS. 3A and 3B depict histograms of the results from screening an Un-Met library (FIG. 3A) and corresponding Pre-Met library (FIG. 3B) for inhibition of the growth of an MRSA strain as described in the Example. The library used in this example was the National Cancer Institute (NCI) Diversity Set V screening library. Separate curves are shown in each panel for unknowns (library compounds), known active antibacterial compounds (known antibacterial agents added to the master plates as controls), and known inactive compounds (the drug metabolism controls added to the master plates).

FIG. 4 is a Venn diagram illustrating Un-Met+Pre-Met Library Screening results. Hit counts associated with the two parallel screens are indicated in the enclosed regions. Hit numbers and descriptions: 1446 wells—UM and PM wells inactive; 39 wells—UM well active, PM well inactive, Parent compound active but activity lost during metabolism (inactive compound metabolite(s)); 42 wells—UM well inactive, PM well active, Parent compound inactive but has at least one active metabolite; 66 wells—UM and PM both active, Parent compound active and not metabolized, or Parent compound active and metabolized to active compound metabolite(s).

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In one aspect, the present disclosure is directed to a pre-metabolized compound library. The pre-metabolized compound library includes a plurality of compound metabolites; and a substrate, wherein the plurality of compound metabolites are selectively distributed in a plurality of wells of the substrate.

As used herein, “pre-metabolized compound” (also interchangeably referred to herein as a “Pre-Met compound” and a “compound metabolite”) refers to a product or products prepared by contacting a compound with a metabolizing agent as described herein. The metabolizing agent converts the compound by a chemical or enzymatic process into possibly one or more compound metabolites. As used herein, “pre-metabolized compound library” refers to a collection of pre-metabolized compounds (compound metabolites) that are selectively distributed in a plurality of wells of a substrate. The pre-metabolized compound library can ultimately be used in high-throughput screening of the compound metabolites.

Suitable substrates include microtiter plates, and any other similar modality for conveniently handling substantial number of compounds such as deep well plates and tube clusters. Microtiter plates suitable as substrate include, for example, 6-well, 24-well, 96-well, 384-well, 1536-well, 3456-well, and 9600-well microtiter plates.

In another aspect, the present disclosure is directed to a method for preparing a pre-metabolized compound library. The method includes providing a compound library comprising a plurality of compounds; and contacting the plurality of compounds with a metabolizing agent to generate a plurality of compound metabolites.

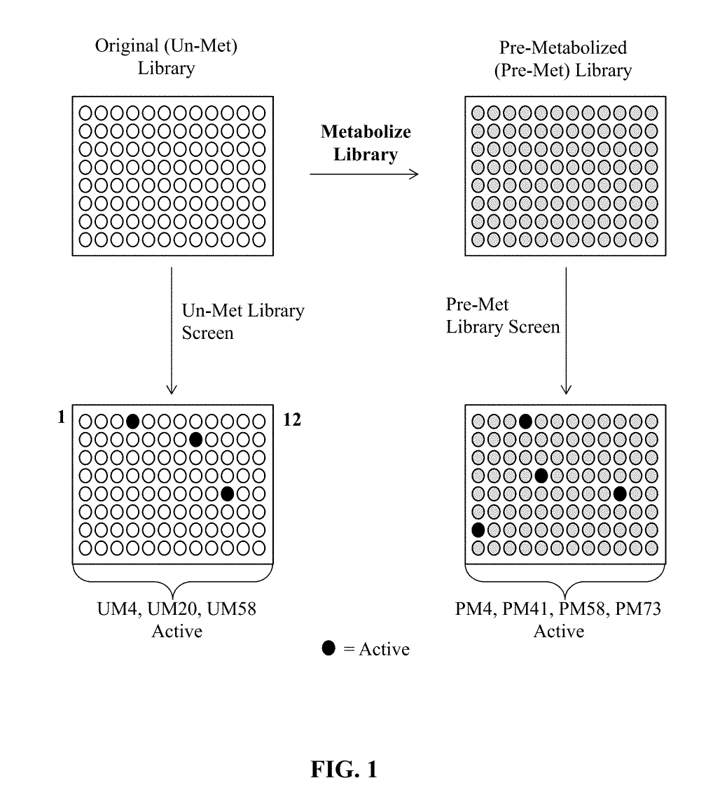

Referring to the schematic illustration of FIG. 1, in the contacting step where the plurality of compounds is contacted with a metabolizing agent, the metabolizing agent (including suitable supporting buffers and reagents) is distributed into each container (well) of a suitable substrate containing a compound library. This results in the formation of a reaction mixture that can be incubated for a period of time such that a plurality of compound metabolites is generated from the original library compounds. The contacting step wherein the compounds of the compound library are incubated with a metabolizing agent can range from less than a minute to days. Suitably, the incubation can be for 24 hours. The incubation can also range from about 5 minutes to about 24 hours. The incubation can occur at any desired temperature. Generally, the incubation occurs at room temperature (about 18° C. to about 22° C.). The incubation can occur more rapidly at temperatures greater than 22° C. Alternatively, the incubation can occur more slowly at temperatures less than 18° C. The concentration of the compound to be metabolized can also be varied from very high (>500 mM) to very low <1 μM. Generally, compound concentrations range from about 0.1 to about 10 mM. Concentrations of the metabolizing agent can also be varied. Generally, for human liver microsomes a concentration of about 0.1 mg/mL of total protein is used, with a possible practical range of about 1 μg/mL to about 1 mg/mL.

As used herein, “metabolizing agent” refers to an agent having an activity that converts a first or starting compound to at least a second compound (the “compound metabolite”). Thus, a compound may be converted into one or more compound metabolites. A compound may remain unmodified (i.e., unmetabolized) by a particular metabolizing agent, or metabolized into one or more primary compound metabolites. These primary compound metabolites can be further converted into additional compound metabolites. Different metabolizing agents can give different compound metabolites and compound metabolite profiles. The degree of conversion, the identify of compound metabolites, and the distribution of compound metabolites will be dependent on the type of metabolizing agent used, its concentration, the time conversion is allowed to proceed, and other factors such as temperature, pH, and the presence of cofactors and other reagents. Without being bound by theory, depending on the incubation time in the contacting step, for example, a short incubation time, the metabolizing agent may convert the first compound to a first compound metabolite. A longer incubation time can result in the conversion of the first compound into a first compound metabolite, which upon prolonged incubation results in conversion to a second compound metabolite. Without being bound by theory, an incubation time can be identified in which the first compound is completely or totally converted to its compound metabolite(s). At any point during the incubation, the compound metabolite(s) can be tested for activity. At any point during the incubation, the reaction mixture including the first compound and the metabolizing agent can be analyzed for conversion of the first compound to its compound metabolite(s).

Suitable metabolizing agents can be chosen from an organ homogenate, a tissue homogenate, cells, cell lysates, cellular fractions, enzymes, an S9 fraction of an organ homogenate, a liver microsomal fraction, and combinations thereof. In another embodiment, suitable metabolizing agents can be selected from the group consisting of an organ homogenate, a tissue homogenate, cells, cell lysates, cellular fractions, enzymes, an S9 fraction of an organ homogenate, a liver microsomal fraction, and combinations thereof.

An organ homogenate can be prepared according to methods known to those skilled in the art. For example, organ tissue can be homogenized in blenders, dounce homogenizers, and other methods for disrupting organ tissue structure. Suitable organs for preparing organ homogenate metabolizing agents include liver, lung, kidney, and intestine.

In one embodiment, suitable cell based metabolizing agents include primary human hepatocytes. In another embodiment, suitable cell based metabolizing agents include immortalized hepatocyte cell lines can be chosen from HepG2 cells, and HepaRG cells, and combinations thereof.

In another embodiment, the metabolizing agent includes cell lysates and cell fractions. Cell lysates and cell fractions can be prepared according to methods known to those skilled in the art. For example, a plurality of cells can be lysed such that the cell membrane of the cells is disrupted such as by freeze-thaw and chemical lysis. A plurality of cells can also be homogenized such that the cell membranes are disrupted. Once the cell membranes are disrupted, cellular fractions can be isolated using methods known to those skilled in the art such as, for example, centrifugation. Suitable cell fractions include endoplasmic reticulum, microsomes, S9 fractions, cytosol and combinations thereof, which contain a variety of metabolic enzymes. In one embodiment, suitable cell fractions can be chosen from endoplasmic reticulum, microsomes, S9 fractions, cytosol and combinations thereof. Endoplasmic reticulum, microsomes, S9 fractions, and cytosol can also be obtained from commercial sources (e.g. ThermoFisher, XenoTech, Sigma-Aldrich, Corning, etc.). Suitable microsomes, S9 fractions, and cytosol cell fractions can be obtained from any species of interest such as, for example, human, canine, primate, mouse, rat, minipig, hamster, guinea pig, fish, etc.

In another embodiment, the metabolizing agent includes an enzyme. Suitable enzymes can be chosen from drug and xenobiotic metabolizing enzymes. Suitable metabolizing enzymes include, for example, aldehyde oxidases, alcohol dehydrogenases, aldehyde dehydrogenases, cytochrome P450s (e.g., CYP1A1/2, CYP2A6, CYP2B6, CYP2C8, CYP2C9, CYP2C19, CYP2D6, CYP2E1, CYP3A4/5, CYP4A11, and combinations thereof), flavin monooxygenases, glutathione S-transferases, monoamine oxidases, sulfotransferases, UDP-glucuronosyltransferases, methyltransferases, acetyltransferases, esterases, amidases, and combinations thereof. Enzymes and enzyme systems can be obtained from any species of interest such as, for example, human, canine, primate, mouse, rat, pig, minipig, hamster, guinea pig, fish, etc. Enzymes can be purified enzyme preparations, crude enzyme preparations, and bacterial, yeast, and animal cells engineered to express drug and xenobiotic metabolizing enzymes (e.g., BACTOSOMES®, commercially available from XENOTECH®, Kansas City, Kans.; and SUPERSOMES®, commercially available from CORNING®, Tewksbury, Mass.), and combinations thereof.

The amount of metabolizing agent contacted with the compound library can vary depending on the particular metabolizing agent used, the degree of conversion to compound metabolites desired, the concentration of the compounds contained in the compound library, the length of time to be used for the compound library metabolization, and combinations thereof. It is within the skill of those skilled in the art to determine the amount of metabolizing agent to contact with the compound to generate compound metabolites.

The method can further include replica plating the compound library. The replica plate of the compound library can serve as a control compound library to which the pre-metabolized compound library is compared.

The method can further include analyzing the plurality of compound metabolites. The plurality of compound metabolites can be subjected to high pressure liquid chromatography (HPLC), mass spectrometry, liquid chromatography tandem mass spectroscopy (LC-MS/MS), gas chromatograph, gas chromatography mass spectroscopy (GC-MS), nuclear magnetic resonance (NMR), and other analytical methods known to those skilled in the art to identify active compounds in the resulting pre-metabolized compound mixtures, and combinations thereof.

As noted above, chemical compound libraries (also referred to herein as “compound libraries” or “chemical libraries” or “libraries”) contain collections of chemical compounds arrayed in a convenient format for desired activity screening in a suitable substrate. Chemical libraries can also be made of several groups of smaller libraries stored in the same location. Chemical compound libraries include custom in house libraries, and commercial and public libraries, as noted above.

As noted above, there are many commercial and public libraries available, for most of which the chemical compound identities are publically known. Selleckchem (Houston, Tex.) offers a number of screening libraries, most of which are targeted towards a specific type of biological activity, ranging in size from <50 compounds (anti-diabetic compound library) to >5000 compounds, including an FDA approved drug library of 1400 compounds and a natural products library of 133 compounds. Maybridge (Leicestershire, United Kingdom) offers diversity oriented screening libraries of 14,000-53,000 compounds. ChemBridge (San Diego, Calif.) offers diversity libraries ranging from 10,000-620,000 compounds. TargetMol (Boston, Mass.) offers a variety of specialized libraries ranging in size from a 41 compound human endogenous ligand library to a 1700 compound approved drug library. A demarcation between simply a set (collection) of individually interesting compounds that can be reasonably studied in a one-at-a time fashion and a “library”, wherein a clear advantage of scale becomes apparent, is unclear. However, given that the smallest libraries are in the 30-200 compound range, and a clear advantage in handling substantial numbers of compounds becomes apparent within this range of compounds, a “library” can reasonably be considered to constitute >30, >50, >80 (the number commonly found in a typical screening plate with the first and last columns left blank for inclusion of control samples), or >96 (the common size of a standard 96 well microtiter plate) compounds arrayed in a suitable substrate (multi-well microtiter plates) format for easy handling.

As known to those skilled in the art, compounds in compound libraries are generally arrayed across wells of the substrate (such as multi-well microtiter plates). Some rows and columns of wells can be left empty for inclusion of control compounds with known activity in the screening assay to be performed or for use as blanks. Compounds contained within the wells are generally solubilized using a solvent such as, for example, dimethyl sulfoxide (DMSO).

In an exemplary embodiment, the present disclosure is directed to screening methods using human liver microsome pre-metabolized libraries. Such an approach has potentially significant advantages over un-metabolized (“Un-Met”) library screening. Most compounds are metabolized into several potentially active metabolites, and the Pre-Met screening approach of the present disclosure thereby offers the potential to significantly increase the number of hits (hit rate) from a library screening effort. Accordingly, the novel approach of the present disclosure will be to significantly increase the hit rate from a library screening effort.

In another aspect, the present disclosure is directed to a method for screening a metabolized compound (Pre-Met) library. The method includes: providing a compound library comprising a plurality of compounds; contacting the plurality of compounds with a metabolizing agent to generate a plurality of compound metabolites; contacting the plurality of compound metabolites with a target; and analyzing the target.

Suitable methods to analyze the target are known to those skilled in the art. For example, when the target is a cell such as an animal cell, the cell can be analyzed for the desirable effects of the compound(s), or its metabolite(s). For example, when the target is a bacterial cell, the bacterial cell can be analyzed for antibacterial activity, for example, by the compound(s), or its metabolite(s). When the target is a diseased cell such as, for example, a cancer cell, the cancer cell can be analyzed for anti-cancer activity (e.g., inhibition of the cancer cell growth, inhibition of the cancer cell division, etc.), for example, by the compound(s), or its metabolite(s). Methods to analyze the target can be designed to identify the molecular target of the pre-metabolized compound(s).

Additionally, when the target is an enzyme or receptor, biochemical assays can be used to analyze for inhibition or otherwise favorable interaction with the enzyme or receptor, for example, by the compound(s), or its metabolite(s).

Suitable metabolizing agents include those described herein.

In an exemplary aspect, the present disclosure is directed to a method for screening candidate compound metabolites for antibacterial activity. The method includes: contacting the candidate antibacterial compounds with human liver microsomes; contacting the mixtures metabolized compounds with a dilute bacterial culture in the presence of a suitable bacterial growth medium; incubating the resulting mixtures for a period of time under conditions favorable to bacterial growth; and analyzing the mixtures for inhibition of bacterial cell growth.

The method can further include separating and/or identifying active compound metabolites. Particularly suitable methods for separating active compound metabolites, which are well known to those skilled in the art, include normal and reverse phase chromatography, ion exchange chromatography, distillation, and sublimation. Active compound metabolites can be identified using methods well known to those skilled in the art, including mass spectroscopy, UV-vis spectroscopy, IR spectroscopy, nuclear magnetic resonance (NMR) spectroscopy, elemental analysis, small molecule X-ray crystallography, melting point determination, chemical derivative/functional group analysis, and combinations thereof. As used herein, “identifying the compound metabolite of the candidate antibacterial compound” refers to determining the chemical structure of the metabolite.

The disclosure will be more fully understood upon consideration of the following non-limiting Example.

### EXAMPLE

### Example 1

In this Example, a whole cell assay was used to demonstrate Pre-Met library screening for anti-MRSA agents.

The National Cancer Institute (NCI) Diversity Set V screening library was obtained from the NCI. This library is comprised of 1593 compounds arrayed in twenty 96-well plates (8 rows×12 columns). Each well contained a library compound at a concentration of 10 mM in 20 μL DMSO. Library compounds are arrayed in columns 2-11 of the plates, with columns 1 and 12 left empty (80 compound maximum per plate).

Known antibacterial agents were added to wells of the first columns of all these NCI master plates as antibacterial activity controls, and known metabolizable drugs were added to wells of the last column of all these NCI master plates as metabolism controls. To the first column (column 1 as illustrated in FIG. 1) of each plate was added, from top to bottom, 20 μL of 10 mM of the following in DMSO: vancomycin, fosfomycin, ampicillin, tetracycline, kanamycin, and chloramphenicol. To the last (bottom) two wells in column 1 were added 20 μL DMSO. To the last column (column 12) of each plate was added, from top to bottom, 20 μL of 10 mM of the following in DMSO: phenacetin, tolbutamide, dextromethorphan, coumarin, cholorozoxazone, and diclofenac. To the last (bottom) two wells in column 12 were added 20 μL DMSO. Plates were stored at -80° C. until further use.

A preliminary antibacterial screening effort indicated that library dilution would result in sufficient hits for further study without also providing an overwhelming number of high MIC hits of less interest. Library samples were first diluted by adding sufficient DMSO to achieve a 4-fold dilution (e.g. for 20 μL this would require addition of 60 μL). This provided a set of Diluted Master/Control Plates with control and library compounds present at 2.5 mM. Plates were stored at −80° C. until further use.

Replica plating was performed with a BIOMEK® 3000 liquid handling workstation (Beckman Coulter, Brea, Calif.) using an 8 channel 20 μL multiple pipetting head. To reduce the number of plates necessary for processing and the volume of samples and reagents required, the Diluted Master/Control Plates were replica plated into multiple 384-well plates to create working copies. The entire library contained in 20×96-well plates were fit into 5×384-well plates. Two copies of the 5×384-well plates, each set containing 4 μL aliquots of samples from the 96-well Diluted Master/Control Plates, were prepared for the screen described below (Copy Plates). DMSO was removed from wells under a strong vacuum (<5 μmHg) for 1-2 days.

The 2 sets of Copy Plates were reconstituted by addition of 4.5 ul of 20% acetonitrile+80% to each well using the BIOMEK® 3000. Plates were placed in a sealed container over wet paper towels and rocked overnight. These Reconstituted Copy Plates were then stored at −80° C.

Next, a copy of the library in its un-metabolized form and a copy of the library in its pre-metabolized form were created. The buffer used for library metabolism was (4×=4 times the concentration of each component in the final solution): 4× KPMEN Buffer: 200 mM KH₂PO₄; 4 mM K₂EDTA; 20 mM glucose-6-phosphate, disodium salt (G6P); 4 mM NADPH, tetrasodium salt; pH adjusted to 7.4 with 1 M KOH. To each well in 1 set of the Reconstituted Copy Plates was added 45 μL 1.1× KEPNM Buffer to give 1 set of unmetabolized (“Un-Met”) Library Plates. To the remaining set was added 45 μL of a freshly prepared mixture of: 1.1× KEPNM Buffer+1 unit/mL glucose-6-phosphate dehydrogenase (G6PDH, for NADPH regeneration)+0.125 mg/mL human liver microsomes (Sekisui XenoTech LLC, Kansas City, Kans., catalog #H0630, Lot #1410013, for compound metabolism) to create 1 set of pre-metabolized (“Pre-Met”) Library Plates. Both sets of plates were incubated at room temperature for 24 hours with gentle rocking to provide 1 set of Un-Met Library Plates and 1 set of Pre-Met Library Plates.

Each well of an Un-Met or Pre-Met Library Plate contained 50 μL of solution containing 200 μM of test agent and/or its metabolites, with each set comprised of 5×384-well plates. From each of these sets was prepared 2 sets of replica plates with 10 μL in each well (Un-Met and Pre-Met Library Test Plates respectively). This allowed both the Un-Met and Pre-Met library screens to be performed in duplicate.

Antibacterial activity screening was performed using methicillin resistant Staphylococcus aureus (MRSA; ATCC # 43300). MH-NCPK selective media used in this Example was comprised of: Mueller Hinton Broth+3% NaCl+8 μg/mL cefoxitin+2 μg/mL polymyxin B+1 μg/mL ketoconazole (MH-NCPK media). A stock of MRSA in MH-NCPK media was prepared such that 10 μL would contain ˜4000 colony forming units (cfu) (MRSA dilute bacterial suspension). To each well in the 2 sets of Un-Met Library Test Plates (five 384-well plates in each set) and the 2 sets of Pre-Met Library Test Plates (five 384-well plates in each set), with wells containing 10 μL of 200 μM compound (Un-Met plates) or compound and compound metabolites (Pre-Met plates) was added 10 μL of the MRSA dilute bacterial suspension. These plates were then incubated at 35° C. for 24 hours to allow bacterial growth. The optical density of each well was then read at 590 nm (0D590) using a TECAN SpectraFluor Plus microtiter plate reader (Artisan Technology Group, Champaign, Ill.). These plates were also read prior to addition of bacteria to identify wells with compounds with a high OD590, so that these well could be examined manually for turbidity post bacterial growth, since a high prior OD590 would interfere with post bacterial growth OD590 measurement.

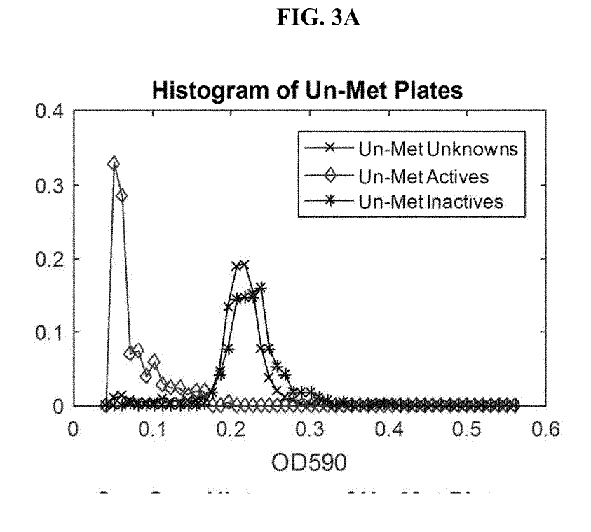

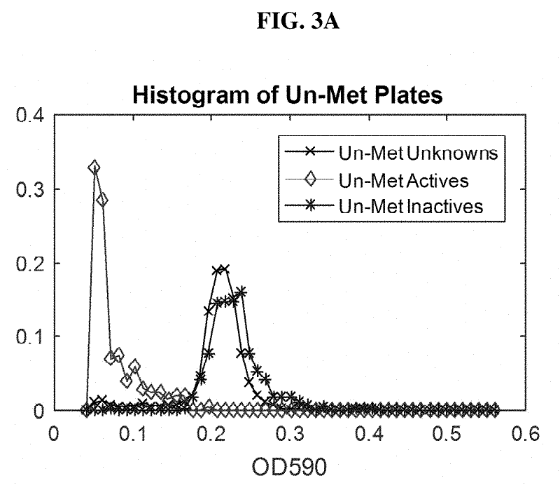

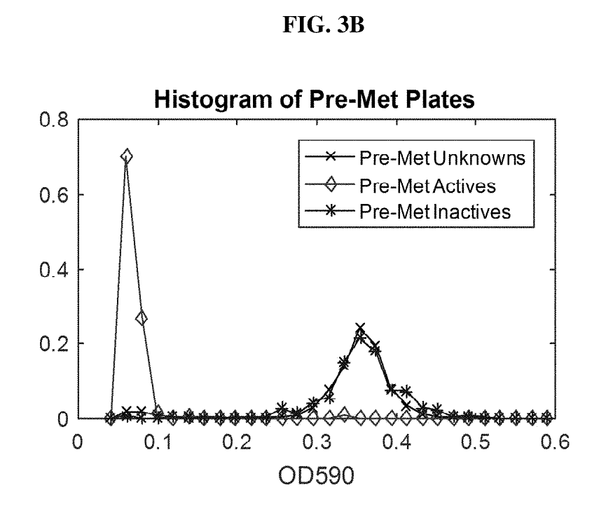

The OD590 optical density data was imported into MATLAB and processed using homemade scripts and programs. The principles for analyzing the results for library screening and selecting hits are well established. Histograms of normalized counts vs OD590 are shown in FIG. 3. Pre-Met samples gave a slightly higher baseline OD590. Curves for known active agents gave the low OD590 peak, and the curves for the known inactive agents (the metabolizable drug controls listed above) gave the high OD590 peaks. In this Example, dextromethorphan had anti-MRSA activity in the Un-Met plates, but not in the Pre-Met plates (since it was metabolized), and was therefore excluded from the list of known inactive agents.

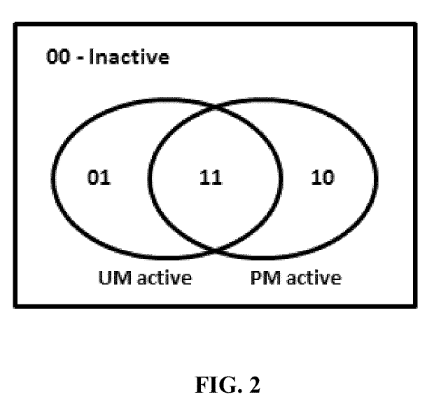

Using a cutoff OD590 of 0.15 for Un-Met plates and 0.18 for Pre-Met plates (below=active, above=inactive), and requiring a given well in both duplicate plates to be below the cutoff for a Un- or Pre-Met well to be counted as a hit. The Un-Met library screen gave 105 hits, and the Pre-Met library screen gave 108 hits. Of these, 39 were unique to the Un-Met library screen (i.e. these agents lost activity upon metabolism), 42 were unique to the Pre-Met library screen (i.e. these were agents which were inactive in their un-metabolized form, and gained activity upon microsomal metabolism), and 66 were common to both sets. A Venn diagram outlining the realm of possibilities for a combined Un-Met and Pre-Met library screen is shown in FIG. 2, and a Venn diagram summarizing the results of the anti-MRSA screening effort described in this example is shown in FIG. 4. These overall screening results demonstrated that library pre-metabolism and screening increased the overall number of below cutoff hits in the anti-MRSA screen by 40%.

Minimal inhibitory concentrations (MICs) were then determined for all Un-Met and Pre-Met hits using a standard broth microdilution approach using 2-fold serial dilutions of active well samples. For any hit in either an Un-Met or Pre-Met well, both corresponding Un-Met and Pre-Met wells were subjected to MIC determination. Aliquots of 10 μL from the appropriate wells in the Un-Met and Pre-Met Library Plates were transferred into the top rows of fresh 384 well plates. Each well of the top 8 wells across the entire plate were then inoculated with 10 μL of MH-NCPK media containing 4000 cfu of the test organism (MRSA). Starting at the top row, 10 μL was transferred into the next row, mixed, and the process continued down the rows until the 7th row, from which 10 μL was removed and discarded after mixing. This provided 7 steps of two-fold dilutions of the original active wells, and a final row (row 8) contained only 10 μL media (blank row). These MIC plates were incubated overnight at 35° C. Turbidity was read (0D590) as described above. The MIC was read as the lowest concentration of test compound for which no turbidity is apparent (transmittance >90% of a media control well). MICs for the overall best 49 hits (lowest MIC between Un-Met and Pre-Met sample of less than or equal to 25 μM) are summarized in Table 2.

TABLE 2
Summary of MICs for Top 49 Hits. Blank cells indicate
no activity (MIC > 100 μM).

MIC (μM)

| Cmpd | Un-Met | Pre-Met |
|---|---|---|
| 1 | 0.78 | 0.78 |
| 2 | | 0.78 |
| 3 | 0.78 | |
| 4 | 50 | 0.78 |
| 5 | 0.78 | 0.78 |
| 6 | 0.78 | 0.78 |
| 7 | 0.78 | 0.78 |
| 8 | 0.78 | 0.78 |
| 9 | | 1.56 |
| 10 | 1.56 | 12.5 |
| 11 | 1.56 | 3.13 |
| 12 | | 3.13 |
| 13 | 3.13 | 50 |
| 14 | 6.25 | 25 |
| 15 | | 6.25 |
| 16 | 6.25 | 12.5 |
| 17 | 25 | 6.25 |
| 18 | 50 | 6.25 |
| 19 | 12.5 | 100 |
| 20 | 12.5 | 12.5 |
| 21 | | 12.5 |
| 22 | 12.5 | |
| 23 | 12.5 | 25 |
| 24 | 12.5 | 50 |
| 25 | 50 | 12.5 |
| 26 | 12.5 | 25 |
| 27 | 12.5 | 25 |
| 28 | 12.5 | 25 |
| 29 | 12.5 | |
| 30 | 12.5 | 12.5 |
| 31 | | 25 |
| 32 | | 25 |
| 33 | | 25 |
| 34 | | 25 |
| 35 | | 25 |
| 36 | 50 | 25 |
| 37 | 25 | |
| 38 | 25 | |
| 39 | 100 | 25 |
| 40 | 25 | 50 |
| 41 | 25 | 100 |
| 42 | 25 | |
| 43 | 25 | 50 |
| 44 | 25 | 25 |
| 45 | 25 | 50 |
| 46 | 25 | |
| 47 | 25 | |
| 48 | 25 | 25 |
| 49 | 25 | |

The results from the MIC determination demonstrate that, for the 49 most active hits overall from this screen, for 24 compounds the Un-Met library sample was more potent than the Pre-Met library sample, which indicates that these compounds were metabolized to less or inactive metabolites. For 9 compounds they were of the same potency, indicating no or little metabolism, or metabolism to a similarly active metabolite. For 16 compounds the Pre-Met sample was more active than the Un-Met sample, indicating metabolism of an inactive or less active precursor to an active or more active metabolite. These 16 compound metabolites represent a 100*16/(24+9)=48% increase in the number of hits on the more potent end of the spectrum of potency obtained in this screening effort, demonstrating the utility of the Pre-Met library screening approach for the identification of novel drug lead and bioactive agents.

The unique Pre-Met hits contain metabolites of Un-Met library compounds with human liver microsome modified chemical structures, which provided a biologically relevant increase in chemical diversity, and which are unlikely to be known compounds. This approach also identifies agents without activity initially but that gained activity upon metabolism, and such compound could serve as pro-drugs of active agents. Further identification of the active metabolites by purification and structure elucidation can provide unique active agents that would be missed without using a Pre-Met library screening approach.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, and “the” are intended to mean that there are one or more of the elements. The terms “comprising”, “including”, and “having” are intended to be inclusive and mean that there may be additional elements other than the listed elements.

What is claimed is:

1. A pre-metabolized compound library comprising:
a plurality of compound metabolites; and
a substrate, wherein the plurality of compound metabolites are selectively distributed in a plurality of wells of the substrate.

2. The pre-metabolized compound library of claim 1, wherein the substrate is a microtiter plate, a deep well plate and a tube cluster.

3. A method for preparing a pre-metabolized compound library, the method comprising:
providing a compound library comprising a plurality of compounds; and
contacting the plurality of compounds with a metabolizing agent to generate a plurality of compound metabolites.

4. The method of claim 3, wherein the metabolizing agent is selected from the group consisting of an organ homogenate, a cell, a cell lysate, a cell fraction, an enzyme, and combinations thereof.

5. The method of claim 4, wherein the cell is selected from the group consisting of a primary hepatocyte, a HepG2 cell, and a HepaRG cell.

6. The method of claim 4, wherein the cell fraction is chosen from endoplasmic reticulum, microsomes, S9 fractions, cytosol and combinations thereof.

7. The method of claim 4, wherein the organ homogenate is a liver homogenate, lung homogenate, kidney homogenate, and intestine homogenate.

8. The method of claim 4, wherein the enzyme is a metabolic enzyme.

9. The method of claim 8, wherein the metabolic enzyme is chosen from a drug metabolizing enzyme, a xenobiotic metabolizing enzyme, and combinations thereof.

10. The method of claim 8, wherein the metabolic enzyme is chosen from aldehyde oxidases, alcohol dehydrogenases, aldehyde dehydrogenases, cytochrome P450s, flavin monooxygenases, glutathione S-transferases, monoamine oxidases, sulfotransferases, UDP-glucuronosyltransferases, methyltransferases, acetyltransferases, esterases, amidases, and combinations thereof.

11. The method of claim 3, further comprising analyzing a plurality of compound metabolites.

12. A pre-metabolized compound library prepared according to the method of claim 3.

13. A method for screening a compound library, the method comprising:
providing a compound library comprising a plurality of compounds;
contacting the plurality of compounds with a metabolizing agent to generate a plurality of compound metabolites;
contacting the plurality of compound metabolites with a target; and
analyzing the target.

14. The method of claim 13, wherein the metabolizing agent is chosen from a cell, a cell lysate, a cell fraction, an enzyme, and combinations thereof.

15. The method of claim 13, wherein the target is chosen from a multicellular organism, a cell, a microorganism, a cellular pathway, an enzyme, a receptor, and combinations thereof.

16. The method of claim 15, wherein the cell is chosen from an animal cell.

17. The method of claim 16, wherein the animal cell is chosen from a cancer cell, a virally-infected cell, a normal cell, a mutated cell, a genetically engineered cell, and combinations thereof.

18. The method of claim 15, wherein the microorganism is chosen from a bacterium, a fungus, a virus, and combinations thereof.

19. The method of claim 13, wherein the target is a cellular pathway.

20. The method of claim 4, wherein the organ homogenate is a liver homogenate, lung homogenate, kidney homogenate, and intestine homogenate.
